(19) United States
(12) Patent Application Publication
(10) Pub. No.: US 2026/0356887 A1
(43) Pub. Date: Nov. 12, 2026

(54) NOVEL MYD88 INHIBITOR FOR TREATMENT OF AUTOIMMUNE DISEASES AND USES THEREOF

(71) Applicant: S&K THERAPEUTICS, Suwon-si, Gyeonggi-do (KR)

(72) Inventors: Sangdun CHOI, Suwon-si (KR); Kevin Jaesuk CHOI, Suwon-si (KR); Mariya FAROOQ, Suwon-si (KR); Bilal Ahmad RATHER, Suwon-si (KR)

(73) Assignee: S&K THERAPEUTICS, Suwon-si, Gyeonggi-do (KR)

(21) Appl. No.: 19/485,690

(22) PCT Filed: May 23, 2024
(86) PCT No.: PCT/KR2024/006955

§ 371 Date: Nov. 19, 2025

(30) Foreign Application Priority Data
May 23, 2023 (KR) 10-2023-0066446

(51) Int. Cl. C07K 7/06; A61K 38/00; A61P 25/28; A61P 29/00; A61P 37/02; C07K 7/08

(52) U.S. Cl. CPC C07K 7/06; A61P 25/28; A61P 29/00; A61P 37/02; C07K 7/08; A61K 38/00; C07K 2319/01

(57) ABSTRACT

An MyD88 inhibitory peptide targeting an MyD88 interaction interface and uses thereof are disclosed. The MyD88 inhibitory peptide MyDIP2-4 effectively inhibits MyD88-mediated signaling in TLR and IL-IR-mediated pathways. MyDIP2-4 specifically interacts with the MyD88 protein in a dose-dependent manner. MyDIP2-4 significantly inhibited IL-23 and Ki-67 in imiquimod-induced psoriasis models, and downregulated cytokines including IL-6 and IFN-γ in EAE. These results suggest that MyDIP2-4 is a promising candidate for the treatment of autoimmune diseases. Therefore, targeting the TIR domain of MyD88 through a reasonable peptide design can provide a new strategy for developing therapeutic agents for immune diseases such as psoriasis and EAE.

### TECHNICAL FIELD

The present invention relates to a peptide that inhibits the MyD88 (myeloid differentiation primary-response 88) signaling pathway, and more particularly, to a peptide that inhibits the MyD88 signaling by targeting the TIR (toll/interleukin-1 receptor) domain of MyD88, a fusion peptide in which the peptide is bound to a cell-penetrating peptide (CPP), a MyD88 inhibitor comprising the peptide or the fusion peptide, and a pharmaceutical composition and cosmetic composition for preventing or treating MyD88-related autoimmune diseases, inflammatory diseases, or neurodegenerative diseases.

### BACKGROUND ART

Toll-like receptor (TLR) signaling is crucial for innate immune defense against external factors (Xiaopei Huang and Yiping Yang, Expert Opin Ther Targets. 2010 August; 14(8): 787-796). TLRs are the most extensively studied pattern recognition receptors (PRRs) that may activate a variety of cells, including immune and non-immune cells. The downstream signaling pathways thereof produce a broad spectrum of immune-stimulating cytokines and chemokines (Ji-Qing Chen, et al., Clinical Reviews in Allergy & Immunology, 2016 February; 50(1):1-17). However, excessive TLR activation impairs immune homeostasis due to the sustained production of pro-inflammatory cytokines, chemokines, and interferons, contributing to the pathogenesis of numerous inflammatory and autoimmune diseases (Wei Gao, et al., Front Physiol. 2017 Jul. 19; 8:508; Li Chen, et al., Acta Pharmacologica Sinica. 2021 November; 42(11):1742-1756).

Myeloid differentiation primary-response 88 (MyD88) is a canonical adaptor protein that links IL-1R (interleukin-1 receptor) and TLR to IRAK (IL-1R-associated kinase) through homotypic protein-protein interactions (Jacques Deguine and Gregory M. Barton, F1000Prime Rep 2014, 6:97). MyD88 serves as an important adapter for IL-1/Toll receptor superfamily signaling. After IL-1R or TLR stimulation, MyD88 is recruited to the activated receptor complex, forms a complex with IRAK4, and activates IRAK1. This activates tumor necrosis factor-associated factor 6, which leads to phosphorylation of IKK (IκB kinase) and activation of NF-κB (nuclear factor kappa light chain enhancer of activated B cells) (O Landgren, N Tageja, Leukemia. 2014 September; 28(9):1799-803). Therefore, MyD88 serves as a central protein in the TLR signaling pathway, functioning as a master switch in various inflammatory diseases. Although MyD88 is considered an important therapeutic target, there are currently no MyD88-targeting drugs approved for clinical use (Jiali Song, et al., Front Pharmacol. 2021 Nov. 29; 12:775117).

MyD88 is a 31-33 kDa adaptor protein with a death domain (DD) at the amino terminus and a TIR domain at the C terminus. The two domains are separated by a short linker region, also called the intermediate domain (ID) (Mark A Olson, et al., Sci Rep. 2015 Sep. 18; 5:14246). MyD88 interacts with IRAK1, IRAK2, IRAK4, and IRAK-M through DD (Su-Chang Lin, et al., Nature. 2010 Jun. 17; 465(7300):885-90). MyD88 is involved in signal transduction initiated by all TLRs except TLR3 (Jacques Deguine and Gregory M. Barton, F1000Prime Rep 2014, 6:97). Recent crystallographic studies have shown that MyD88 TIR (MyD88^{TIR}) forms a higher-order assembly composed of two parallel strands of TIR domains arranged head-to-tail, with subunits constituting each strand (Max T B Clabbers, et al., Nat Commun. 2021 May 10; 12(1):2578). During the nucleation and elongation steps of MyD88^{TIR} assembly formation, incoming MyD88^{TIR} molecules bind to MyD88^{TIR} subunits at the BB surface (BB loop site) or EE surface (αE helix site) of MAL^{TIR} (MyD88 adaptor-like protein TIR); interstrand interactions through the BC (αB and αC helices) and CD (CD loop and αD helix region) surfaces induce conformational changes in the αB helix and BB loop of the TIR domain molecules, which enable interaction with the EE surface of the newly incoming MyD88^{TIR} subunit. MAL-induced MyD88^{TIR} crystallite formation in the solution was found to be disrupted by alanine substitutions at the intrastrand interface of R196, D197, P200, W284, and R288 and at the interstrand interface of K238, L241, S266, and R269 (Thomas Ve, et al., Nat Struct Mol Biol. 2017 September; 24(9):743-751). Additionally, intrastrand mutations of R196A, W284A, I253D, and R288A were shown to interfere with TLR4 ligand lipopolysaccharide (LPS)-induced NF-κB activation, while P200A in the B loop significantly reduced activation (Max T B Clabbers, et al., Nat Commun. 2021 May 10; 12(1):2578). In the central nervous system (CNS), activation of MyD88-dependent receptors, TLRs, has been implicated in the pathogenesis of multiple sclerosis (MS) and experimental autoimmune encephalomyelitis (EAE) (Chao Zheng, et al., Front Mol Neurosci. 2020 Jan. 10; 12:314). In the CNS, TLR activation induces the production of proinflammatory cytokines and chemokines that contribute to the demyelination and neurodegeneration observed in MS and EAE.

MS is the most common non-traumatic disability in young adults and is characterized by CNS lesions due to perivascular immune cell infiltration and destruction of myelin, oligodendrocytes, and neurons (Socorro Miranda-Hernandez, Alan G Baxter, Am J Clin Exp Immunol. 2013 Feb. 27; 2(1):75-93). MS occurs in young adults with complex genetic traits influencing susceptibility and may be triggered by inflammatory environmental insults, such as viral infections (R Dobson, G Giovannoni, Eur J Neurol. 2019 January; 26(1):27-40). Activation of CD4⁺ (cluster of differentiation 4+) autoreactive T cells and development into a Th1 (T helper 1) phenotype are important processes in the early stages of the disease. These cells are likely to play an important role in long-term progression. Damage to the target tissue, the CNS, may be mediated by other immune system components such as antibodies, complement, CD8⁺ T cells, and innate immune cell-produced proteins. Changes in the immunoregulatory network, including Th2 cells, regulatory CD4⁺ T cells, and NK (natural killer) cells, may contribute to the relapsing-remitting or chronic progressive nature of the disease (Mireia Sospedra, Roland Martin, Annu Rev Immunol. 2005; 23:683-747). The experimental model used in MS is EAE. EAE is a complex disorder in which a combination of numerous immunopathological and neuropathological mechanisms resembles the core pathological features of MS such as inflammation, demyelination, axonal loss, and gliosis (Cris S Constantinescu, et al., Br J Pharmacol. 2011 October; 164(4):1079-106). The role of TLRs in MS and EAE has been extensively studied (Chao Zheng, et al., Front Mol Neurosci. 2020 Jan. 10; 12:314).

Various autoimmune and autoinflammatory disorders, including psoriasis, are associated with activation of nucleic acid-detecting TLRs by self-nucleic acids (Nicholas A Lind, et al., Nature Reviews Immunology, 2022 April; 22 (4):224-235). Psoriasis is a widespread, chronic inflammatory skin disease characterized by increased cell proliferation, altered epidermal differentiation, and inflammatory cell infiltration, the pathogenesis of which depends on genetic predisposition and environmental factors (Philipp Novoszel, et al., EMBO Mol Med. 2021 Apr. 9; 13(4):e12409). Psoriasis is considered a T-cell-driven disease because T-cell-derived cytokines such as IL17A and IL22 induce keratinocytes to hyperproliferate and differentiate in an abnormal manner, forming psoriatic plaques. Nonetheless, PRR (pattern recognition receptor)-mediated recognition of danger-associated molecular patterns (DAMPs) or pathogen-associated molecular patterns (PAMPs) and the innate immune response of keratinocytes or plasmacytoid dendritic cells (pDCs) are considered to be early initiating events in psoriasis that promote adaptive and autoimmune responses. Keratinocytes may produce proinflammatory cytokines to initiate an inflammatory T cell phenotype in psoriasis following DAMP or PAMP stimulation. Targeting PRR-mediated innate immune activation of keratinocytes or pDCs in combination with T cell treatment may be an effective treatment option for psoriasis (Lixiang Sun, et al., J Immunol Res. 2019 Nov. 14; 2019:1824624). The role of TLRs in psoriasis has been studied, and anti-IL-12/IL-23 agents as well as antagonists of TLR7 and TLR8 have shown potential in the treatment of psoriasis (Farzaneh Rahmani, Nima Rezaei Rahmani, Expert Rev Clin Immunol. 2016 December; 12(12):1289-1298). Inhibition of MyD88 has been shown to attenuate imiquimod (IMQ)-induced skin inflammation (Hanitriniaina Rabeony, et al., Eur J Immunol. 2015 October; 45(10):2847-57).

The present invention was intended to develop a novel peptide that inhibits MyD88-mediated signaling by targeting the MyD88 TIR domain. To this end, a novel peptide was designed and fused with a cell-penetrating peptide such as penetratin to enhance cellular uptake. The efficacy of the produced peptides was evaluated for the ability to inhibit MyD88-dependent signaling and TLR- and IL-1R-induced cytokine activation in mouse and human macrophages. In addition, the therapeutic potential of the most potent peptide in both IMQ-induced psoriasis models and EAE was evaluated, and the mechanism of action thereof was investigated. These results demonstrate that rationally designed peptides are potent inhibitors of MyD88-mediated signaling, thereby providing a promising strategy for the development of therapeutics for autoimmune diseases.

The above information described in this Background section is only for improving the understanding of the background of the present invention, and therefore it may not include information that form the prior art that is already known in this country to a person of ordinary skill in the art.

### SUMMARY OF THE INVENTION

An object of the present invention is to provide a peptide having the function of inhibiting the MyD88 signaling pathway.

Another object of the present invention is to provide a fusion peptide in which the peptide is bound to a cell-penetrating peptide (CPP).

Still another object of the present invention is to provide a MyD88 inhibitor comprising the peptide or the fusion peptide.

Yet another object of the present invention is to provide a pharmaceutical composition for preventing or treating a MyD88-related autoimmune disease, an inflammatory disease, or a neurodegenerative disease, comprising the peptide or the fusion peptide.

Still yet another object of the present invention is to provide a method of preventing or treating a MyD88-related autoimmune disease, an inflammatory disease, or a neurodegenerative disease using the peptide or the fusion peptide, the use of the peptide or the fusion peptide for the prevention or treatment of a MyD88-related autoimmune disease, an inflammatory disease, or a neurodegenerative disease, and the use of the peptide or the fusion peptide for the manufacture of a medicament for the prevention or treatment of a MyD88-related autoimmune disease, an inflammatory disease, or a neurodegenerative disease.

Even yet another object of the present invention is to provide a cosmetic composition for preventing or ameliorating a MyD88-related autoimmune disease, an inflammatory disease, or a neurodegenerative disease, comprising the peptide or the fusion peptide.

In order to accomplish the above objects, the present invention provides a peptide represented by any one amino acid sequence selected from the group consisting of SEQ ID NO: 1 to SEQ ID NO: 11.

The present invention also provides a fusion peptide in which the peptide is bound to a cell-penetrating peptide (CPP).

The present invention also provides a MyD88 inhibitor comprising the peptide or the fusion peptide.

The present invention also provides a pharmaceutical composition for preventing or treating a MyD88-related autoimmune disease, an inflammatory disease, or a neurodegenerative disease, comprising the peptide or the fusion peptide.

The present invention also provides a method of preventing or treating a MyD88-related autoimmune disease, an inflammatory disease, or a neurodegenerative disease, comprising administering the peptide or the fusion peptide.

The present invention also provides the use of the peptide or the fusion peptide for the prevention or treatment of a MyD88-related autoimmune disease, an inflammatory disease, or a neurodegenerative disease.

The present invention also provides the use of the peptide or the fusion peptide for the manufacture of a medicament for the prevention or treatment of a MyD88-related autoimmune disease, an inflammatory disease, or a neurodegenerative disease.

The present invention also provides a cosmetic composition for preventing or ameliorating a MyD88-related autoimmune disease, an inflammatory disease, or a neurodegenerative disease, comprising the peptide or the fusion peptide.

### BRIEF DESCRIPTION OF THE DRAWINGS

FIG. 1 shows a computational design of MyD88 inhibitory peptides, (a) showing a MyD88 assembly model highlighting specific regions targeted for peptide design, in which the toll/interleukin-1 receptor (TIR) domain of MyD88 oligomerizes to form a signaling complex, which triggers downstream pathways leading to proinflammatory cytokine production, (b-g) showing specific regions of the MyD88^{TIR} domain interface identified for computational design of the inhibitory peptides, in which these regions include the MyDIP2-4-derived αE helix (b), DD loop (c), RE+EE loop (d), αC helix (e), αC′ helix (f), and BB loop (g), and the peptides were designed to inhibit MyD88 signaling and prevent the production of proinflammatory cytokines.

FIG. 2 schematically shows the screening and identification of potential MyD88 inhibitory peptides, (a and b) showing graphs illustrating results of quantification of TNF-α and IL-6 secreted by mouse macrophages pretreated with the indicated concentrations of peptides and stimulated with LPS (100 ng/ml), (c) showing a graph illustrating results of cell viability analysis (3-(4,5-dimethylthiazol-2-yl)-2,5-diphenyltetrazolium bromide, MTT) of mouse macrophages treated with candidate peptides at the indicated concentrations, (d) showing results of evaluation of the inhibitory effect of MyDIP2 on the MyD88-mediated signaling pathway by measuring TNF-α levels in RAW 264.7 cells using ELISA, in which the cells were pretreated with MyDIP2 peptide at concentrations of 1, 10, and 50 μM, and then activated with various concentrations of Pam₃CSK₄ (acting on TLR2/1), FSL-1 (TLR2/6), poly(I:C) (TLR3), flagellin (Flg, TLR5), imiquimod (IMQ, TLR7), R848 (TLR7/8), TL8 (TLR8), or CpG oligodeoxynucleotide (CpG-ODN, acting on TLR9), results are represented as mean±SD, *p<0.33, **p<0.002, ***p<0.001, MTT is 3-(4,5-dimethylthiazol-2-yl)-2,5-diphenyltetrazolium bromide; mTNF-α is mouse tumor necrosis factor alpha; mIL-6 is mouse interleukin-6; LPS is liposaccharide; NF-κB is nuclear factor kappa B; MAPK is mitogen-activated protein kinase; IC₅₀ is half-maximal inhibitory concentration; and IMQ is imiquimod.

FIG. 3 shows the selection of the optimal peptide among derivatives, (a) showing a graph illustrating results of cell viability analysis using MTT assay after treatment with 50 μM candidate peptide, (b) showing a graph illustrating the level of TNF-α secretion after pretreatment with 20 μM peptide and then treatment with LPS (100 ng/ml), (c) showing a graph illustrating that MyDIP2-4 exerts a dose-dependent effect on LPS-induced TNF-α secretion, (d) showing an IC₅₀ graph, (e) showing a graph illustrating the effect of MyDIP2-4 on LPS-induced IL-6 secretion, (f) showing results of Western blot analysis of IMQ-induced secretion of NF-κB and MAPKs after MyDIP2-4 treatment, in which RAW 264.7 cells were treated with MyDIP2-4 in the presence or absence of 2 μg/ml IMQ, and Western blot analysis of p-ERK, T-ERK, JNK, p-JNK, pp-38, pp-65, and Iκ-Bα was performed, with β-actin serving as a loading control, (g) showing a graph illustrating the dose-dependent effect of MyDIP2-4 on various concentrations of TLR ligands Pam₃CSK₄ (TLR1/2), FSL-1 (TLR2/6), poly(I:C) (TLR3), flagellin (TLR5), resiquimod (R848, TLR7/8), TL8 (TLR8), or CpG-ODN (TLR9), in which results are represented as mean±SD, *p<0.33, **p<0.002, ***p<0.001, MTT is 3-(4,5-dimethylthiazol-2-yl)-2,5-diphenyltetrazolium bromide; TNF-α is tumor necrosis factor alpha; LPS is liposaccharide; NF-κB is nuclear factor kappa B; MAPKs are mitogen-activated protein kinases; IC₅₀ is half-maximal inhibitory concentration; and IMQ is imiquimod.

FIG. 4 shows the effect of MyDIP2-4 on TLR-mediated activation of human macrophages, MOG₃₅₋₅₅ (myelin oligodendrocyte glycoprotein peptide fragment 35-55)-induced EAE, and the biophysical interactions between MyDIP2-4 and MyD88 proteins, (a) showing a graph illustrating the effects of 20 μM MyDIP2 and MyDIP2-4 on the viability of THP-1-derived macrophages as determined using MTT assay, (b and c) showing graphs illustrating the secretion of the cytokine TNF-α measured using ELISA to evaluate dose-dependent inhibition by MyDIP2 and MyDIP2-4, respectively, in response to TLR4 stimulation by LPS (100 ng/ml), (d) showing results of evaluation of TNF-α secretion using ELISA after THP-1-derived macrophages were pretreated with MyDIP2-4 peptide and stimulated with IL-1β (150 ng/ml), (e) showing a graph illustrating IL-1β secretion in response to NLRP3 (NACHT, LRR, and PYD domains-containing protein 3) activation in the presence or absence of MyDIP2-4, (f) showing the interaction between MyDIP2-4 and MyD88 proteins, where the sensorgram depicts the binding of MyDIP2-4 to MyD88 protein in a dose-dependent manner, and the sensorgram shows real-time changes in response units over time, indicating the strength and dynamics of MyDIP2-4-MyD88 interactions, (g) showing that mice were immunized with MOG₃₅₋₅₅ peptide in CFA (complete Freund's adjuvant) and administered pertussis toxin twice, once on day 0 and again 24 hours later to test the therapeutic potential of MyDIP2-4 in the progression of EAE, (h) showing clinical scores calculated according to the protocol provided by the kit manufacturer, and (i) showing the body weight of mice in each group measured on the indicated date, with graphs showing results of ELISA to measure the levels of (j) IL-17, (k) TNF-α, (1) IFN-γ, and (m) IL-6 in plasma samples separated from the blood by centrifugation at 3000 RPM, in which results are represented as mean±SD, *p<0.33, **p<0.002, ***p<0.001, MTT is 3-(4,5-dimethylthiazol-2-yl)-2,5-diphenyltetrazolium bromide; TLR is toll-like receptor; TNF-α is tumor necrosis factor alpha; LPS is liposaccharide; IL is interleukin; ELISA is enzyme-linked immunosorbent assay; and MOG is myelin oligodendrocyte glycoprotein.

FIG. 5 shows the effect of MyDIP2-4 on IMQ-induced psoriasis, (a) showing that psoriasis was induced by topical application of 5% IMQ onto the shaved back of C57BL/6 mice, dexamethasone (10 mg/kg) was selected as a positive control group, the therapeutic effect of MyDIP2-4 was evaluated at two doses, 5 mg/kg and 20 mg/kg, and on day 6, the mice were euthanized and further experiments were performed, (b) showing photographs of the back of the mice on the last day before euthanasia, (c) showing a graph illustrating PASI scores of drug-treated and IMQ-treated mice to assess disease severity, (d) showing a graph illustrating the effects of MyDIP2-4 and dexamethasone on the body weight of IMQ-induced psoriasis mice, (e) showing results of H&E staining of the epidermis to evaluate the epidermal thickness in different groups, and (f) showing results of immunohistochemical analysis of KI-67, IL-23, and IL-17 expression in skin tissue, in which results are represented as mean±SD, *p<0.33, **p<0.002, ***p<0.001, IMQ is imiquimod; PASI is psoriasis area and severity index; and H&E is hematoxylin and eosin.

### DETAILED DESCRIPTION OF THE INVENTION AND PREFERRED EMBODIMENTS OF THE INVENTION

Unless otherwise defined, all technical and scientific terms used herein have the same meanings as typically understood by those skilled in the art to which the present invention belongs. In general, the nomenclature used herein is well known in the art and is typical.

MyD88 is called one of the pillars of immunology. MyD88 is a ubiquitously expressed cytoplasmic adaptor protein that plays a central role in the TLR and IL-1R signaling pathways. The TLR/IL-1R pathway governs the proliferation and development of cells of the innate and adaptive immune system (Lingfeng Chen, et al., J Med Chem. 2020 Nov. 25; 63(22):13316-13329). Because of the central role in various pathways involved in the immune response, MyD88 is an attractive target for controlling immune responses in various diseases, including autoimmune diseases (Jiali Song, et al., Front Pharmacol. 2021 Nov. 29; 12:775117). The TIR domain of MyD88 plays a crucial role in recruiting various signaling proteins and initiating downstream signaling cascades.

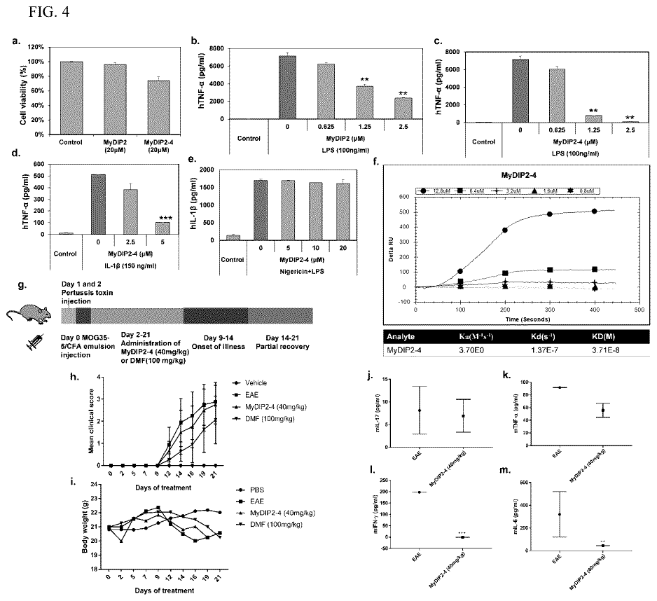

Development of MyD88 interface-binding peptides provides a promising approach to modulate MyD88-mediated signaling by specifically targeting protein-protein interactions at the MyD88 interface, thereby inhibiting downstream signaling and reducing inflammation and other disease symptoms. Designing peptides targeting the TIR domain of MyD88 may effectively inhibit MyD88-dependent signaling in TLR- and IL-1R-mediated signaling pathways, making it an important potential therapeutic target for autoimmune diseases. The present inventors produced peptides targeting the interaction interface of MyD88 using the PyRosetta program (Chaudhury, S. et al., Bioinformatics 2010 Mar. 1; 26(5):689) and evaluated the efficacy of the MyDIP2-4 peptide in inhibiting TLR and IL-1R signaling in mouse and human macrophages. Although MyD88 is not involved in TLR3 signaling, the fact that the TIR domain of MyD88 is also present in TRIF (TIR-domain-containing adapter-inducing interferon-β) explains the peptide's effect on TLR3-mediated TNF-α, as the peptide was designed to target the TIR domain. Ka and Kd values obtained from SPR (surface plasmon resonance) analysis indicate that MyDIP2-4 strongly interacts with MyD88 (FIG. 4f), and with an IC₅₀ value of 2.6 μM, MyDIP2-4 is promising compared to other available MyD88-inhibiting peptides (Shira Dishon, et al., Sci Rep. 2018 Jun. 21; 8(1):9476).

The relationship between MyD88 and NLRP3 has long been debated. Recently, it was reported that activation of the NLRP3 inflammasome inhibits TLR signaling and cytokine production induced by agents or infections through caspase-1 proteolytic cleavage of MyD88 (Monika Avbelj, et al., Front Immunol. 2022 Jan. 5; 12:790258). NLRP3 is downregulated by MyD88 inhibition, but MyD88 inhibition has not been shown to directly inhibit NLRP3-mediated IL-1β secretion (Vivek Verma, et al., Scand J Immunol. 2021; 94:e13104). This study is consistent with the results of the present invention in which no effect of the MyDIP2-4 peptide was observed when NLRP3 was activated by nigericin (FIG. 4e).

Inhibition of MyD88-mediated signaling may have therapeutic benefits for diseases such as psoriasis, an inflammatory skin disease involving both the innate and adaptive immune systems. The pathophysiology of psoriasis is complex. Despite the clinical efficacy of etanercept, a TNF-α blocker, in the treatment of psoriasis, the treatment of psoriasis remains a significant challenge. The PASI score is based on the degree and area of erythema, induration, and desquamation of plaques from other parts of the body.

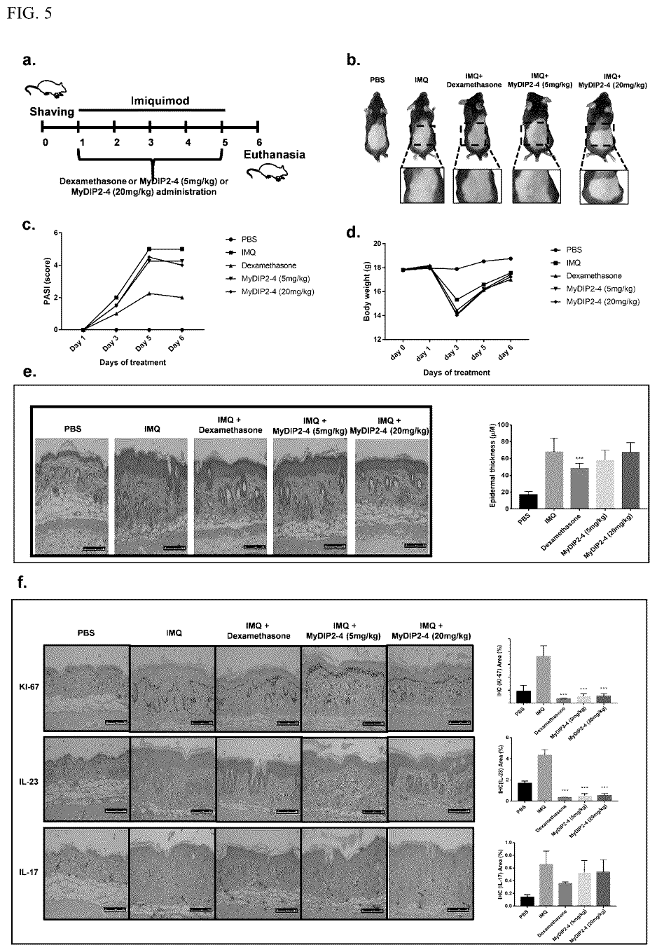

The pathophysiology of psoriasis involves activation of dendritic cells, and IL-23 is required for disease progression (Tom C Chan, et al., Ther Adv Chronic Dis. 2018 May; 9(5):111-119). High levels of IL-23 are associated with erythema, mixed inflammatory infiltrates, and epidermal proliferation (Leslie van der Fits, et al., The Journal of Immunology. 2009 May 1; 182 (9):5836-45). The present inventors aimed to investigate the effect of MyDIP2-4 on psoriasis models. Immunohistochemical analysis showed that both doses of MyDIP2-4 significantly reduced IL-23 levels in skin tissue (FIG. 5f), which is consistent with previous findings (Fenli Shao, et al., Biochem Pharmacol. 2016 Sep. 1; 115:94-103).

IL-23 plays an important role in the pathogenesis of psoriasis by promoting the production of IL-17 from γδ T cells. Subsequent signaling through IL-17 is associated with the development of neutrophilic microabscesses, hyperproliferation, and attenuation of keratinocyte differentiation, resulting in epidermal hyperplasia at the site of injury (Hye-Lin Ha, et al., Proc Natl Acad Sci USA. 2014 Aug. 19; 111(33):E3422-31). MyDIP2-4 significantly reduced IL-23 levels, but did not alter IL-17 levels in skin tissue (FIG. 5f). These results may be due to the fact that IL-17 production may be either IL-23 dependent or independent, as previously reported in the literature (Y Yoshiga, et al., Int J Mol Med. 2008 September; 22(3):369-74.).

IL-17 has been demonstrated to upregulate KI-67 (Hanna Niehues, et al., JID Innov. 2021 Oct. 22; 2(1):100066), a well-established marker of cell proliferation that correlates with the clinical severity of psoriasis and is upregulated in psoriatic skin lesions (Y Luo, et al., Clin Exp Immunol. 2020 October; 202(1):1-10). Although IL-17 was not downregulated, KI-67 was significantly downregulated in MyDIP2-4-treated mouse skin tissues compared to IMQ-treated mouse tissues. MyD88 expression is positively correlated with high KI-67 expression (Jing Niu, et al., Mol Med Rep. 2020 August; 22(2):1243-1256), demonstrating that MyDIP2-4 downregulated MyD88 expression and thus also reduced KI-67 expression. Notably, MyDIP2-4 treatment did not significantly reduce epidermal thickness (FIG. 5f), which is consistent with the results of a study by Costa et al. (Sara Costa, et al., J Leukoc Biol. 2017 September; 102(3):791-803).

MS is a chronic neuroautoimmune and neurodegenerative disease that causes CNS inflammation, demyelination, and axonal and neuronal cell death. EAE is a widely used animal model for MS research (Cris S Constantinescu, et al., Br J Pharmacol. 2011 October; 164(4):1079-106). EAE is induced by injecting components of the immune system that induce an immune response against the myelin sheath of test animals. This results in neurological symptoms similar to those observed in MS patients. However, the exact cause of MS has not yet been fully elucidated (Bert A't Hart, et al., EBioMedicine. 2021 June; 68:103392). Although the causative agents of MS and EAE may be different, the EAE model has provided important information about the basic mechanisms of MS and is being used to develop and test new treatments for the disease (Andrew P Robinson, et al., Handb Clin Neurol. 2014; 122:173-89).

The therapeutic efficacy of targeting MyD88 for the treatment of EAE is of current interest. One study showed that treatment with a MyD88 inhibitor reduced proinflammatory cytokine production and alleviated EAE symptoms in mice (Shira Dishon, et al., Front Immunol. 2017 May 29; 8:615).

Accordingly, an aspect of the present invention relates to a peptide represented by any one amino acid sequence selected from the group consisting of SEQ ID NO: 1 to SEQ ID NO: 11.

As used herein, the term “peptide” refers to a linear molecule formed by binding amino acid residues to each other by a peptide bond. The peptide may be prepared according to a chemical synthesis method known in the art.

The peptide is a peptide targeting the interaction interface of MyD88, and is defined herein as MyDIP. The amino acid sequences of MyDIP1 to MyDIP7 and derivatives of MyDIP2, such as MyDIP2-1 to MyDIP2-4, are as follows.

MyDIP1:
(SEQ ID NO: 1)
WFWTRLAKA
MyDIP2:
(SEQ ID NO: 2)
WFWTRLAKALSLP
MyDIP3:
(SEQ ID NO: 3)
KYKAMKKE
MyDIP4:
(SEQ ID NO: 4)
DYTNPCTKS
MyDIP5:
(SEQ ID NO: 5)
SDDYLQSK
MyDIP6:
(SEQ ID NO: 6)
ECDFQTMFALSL
MyDIP7:
(SEQ ID NO: 7)
SDRDVLPGTCVWSIA
MyDIP2-1:
(SEQ ID NO: 8)
WFWYRLAKKLSLP
MyDIP2-2:
(SEQ ID NO: 9)
WFWYRLIKKLSLP
MyDIP2-3:
(SEQ ID NO: 10)
WFWYILLKKLSLP
MyDIP2-4:
(SEQ ID NO: 11)
WLWFILFKKLSLP

The peptide according to the present invention is to be understood as encompassing variants in which an amino acid residue is conservatively substituted at a specific amino acid residue position, or fragments thereof.

As used herein, “conservative substitution” refers to a modification that includes substituting one or more amino acids with amino acids having similar biochemical properties that do not cause loss of biological or biochemical functions of the peptide.

A “conservative amino acid substitution” is a substitution in which an amino acid residue is replaced with an amino acid residue having a similar side chain. Classes of amino acid residues with similar side chains have been defined in the art and are well known. These classes include amino acids with basic side chains (e.g., lysine, arginine, histidine), amino acids with acidic side chains (e.g., aspartic acid, glutamic acid), amino acids with uncharged polar side chains (e.g., glycine, asparagine, glutamine, serine, threonine, tyrosine, cysteine), amino acids with nonpolar side chains (e.g., alanine, valine, leucine, isoleucine, proline, phenylalanine, methionine, tryptophan), amino acids with beta-branched side chains (e.g., threonine, valine, isoleucine), and amino acids with aromatic side chains (e.g., tyrosine, phenylalanine, tryptophan, histidine).

It is expected that the MyDIP1 to MyDIP7 and MyDIP2-1 to MyDIP2-4 peptides of the present invention may still retain activity even when they have conservative amino acid substitutions.

The peptide according to the present invention is to be understood as encompassing peptides having substantially the same function and/or effect as the peptide and having an amino acid sequence homology of 80% or 85% or more, preferably 90% or more, more preferably 95% or more, most preferably 99% or more with the amino acid sequence of SEQ ID NO: 1 to SEQ ID NO: 11, but is not limited thereto.

In the present invention, the peptide may be characterized by inhibiting MyD88-mediated signaling and thereby suppressing TLR- and IL-1R-mediated signaling pathways.

In the present invention, the term “TLR-mediated signaling pathway” refers to a signaling pathway through TLR, which may be a reaction dependent on a complex formed by TLR and the adaptor protein MyD88 or TLR and the adaptor protein TRIF (in the case of TLR3), through which a signal is transmitted. Activated TLRs activate NF-κB through a MyD88-dependent signaling pathway, translocating the same to the nucleus and inducing the activation of MAPK. Activation of the NF-κB and MAPK causes the secretion of proinflammatory cytokines such as TNF-α and IL-6.

In the present invention, the term “TIR domain” is a domain for intracellular signaling, has three highly conserved regions, and mediates interactions between TLRs and other signaling molecules.

Another aspect of the present invention relates to a fusion peptide in which the peptide is bound to a cell-penetrating peptide (CPP).

In the present invention, the term “cell-penetrating peptide (CPP)” is a type of signal peptide, which is a peptide that is a combination of specific amino acid sequences used for the purpose of delivering macromolecules such as proteins, DNA, and RNA into cells. To date, CPPs have been employed for intracellular delivery of various small molecules and macromolecules such as proteins, peptides, RNA, and DNA. Most cell-penetrating peptides are derived from protein transduction domains (PTDs) or membrane-translocating sequences, and unlike general cellular uptake pathways of external substances, the cell-penetrating peptides are expected to play a groundbreaking role by entering cells without damaging the cell membrane and enabling the intracellular delivery of even DNA and proteins, which are known to be unable to cross the cell membrane.

The fusion peptide of the present invention utilizes a cell-penetrating peptide. The cell-penetrating peptide is not particularly limited so long as it has the characteristic of entering cells through a cell endocytosis mechanism, but is preferably selected from the group consisting of cell-penetrating peptides listed in Table 1 below, and variants thereof.

TABLE 1

| | | | SEQ IO |
|---|---|---|---|
| Peptide | References | Sequence | NO: |
| Penetratin | Derossi et al., (1994) J. | RQIKIWFQNRRMKWKK | 12 |
| | Biol. Chem. 269(14), 10444- | | |
| | 10450 | | |
| TAT | Takeshima et al., (2003) J. | YGRKKRRQRRR | 13 |
| | Biol. Chem. 278(2), 1310- | | |
| | 1315 | | |
| TAT₍₄₈₋₆₀₎ | Dennison et al., (2007) | GRKKRRQRRRPPQ | 14 |
| | Biochem. and Biophy. Res. | | |
| | Comm. 363, 178 | | |
| TAT₍₄₉₋₅₇₎ | Baoum et al., (2012) Int. J. | RKKRRQRRR | 15 |
| | Pharm. 427, 134-142 | | |
| R8 | Chu et al., (2015) | RRRRRRRR | 16 |
| | Nanotechnol. Biol. Med. 11, | | |
| | 435-446 | | |
| R9-TAT | Futaki et al., (2001) J. | GRRRRRRRRRPPQ | 17 |
| | Biol. Chem. 276, 5836-5840 | | |
| Pep-1 | Deshayes et al., (2008) | KETWWETWWTEWSQPKKKRKV | 18 |
| | Advanced Drug Delivery | | |
| | Reviews, 60, 537-547 | | |
| Hph-1 | Choi et al., (2006) Nat. | YARVRRRGPRR | 19 |
| | Med. 12, 574-579. | | |
| MAP | Wada et al., (2013) Bioorg. | KLALKLALKALKAALKLA | 20 |
| | Med. Chem. 21, 7669-7673 | | |
| pVEC | Elmquist et al., (2006) | LLIILRRRIRKQAHAHSK | 21 |
| | Biochim. Biophys. Acta. | | |
| | 1758, 721-729 | | |
| MPG | Simeoni, (2003) Nucleic | GALFLGFLGAAGSTMGAWSQPKK | 22 |
| | Acids Res. 31, 2717-2724 | KRKV | |
| Transportan | Pae et al., (2014) J. | GWTLNSAGYLLGKINLKALAALA | 23 |
| | Controlled Release, 192, | KKIL | |
| | 103-113 | | |
| gH625 | Galdiero et al., (2015) | HGLASTLTRWAHYNALIRAF | 24 |
| | Biochim. Biophys. Acta (BBA) | | |
| | Biomembr. 1848, 16-25. | | |
| VP22 | Elliott and O'Hare (1997) | NAKTRRHERRRKLAIER | 25 |
| | Cell, 88, 223-233 | | |

In one embodiment of the present invention, an experiment was conducted by selecting penetratin represented by the amino acid sequence of SEQ ID NO: 12, and it will be apparent to those skilled in the art that, even when a cell-penetrating peptide other than the cell-penetrating peptide actually used is fused with the peptide of the present invention, an effect similar to that of the present invention is exhibited.

In the present invention, the peptide and the cell-penetrating peptide may be linked directly or through a linker.

In one embodiment of the present invention, a peptide represented by any one amino acid sequence selected from the group consisting of SEQ ID NO: 1 to SEQ ID NO: 11 exhibited an effect of inhibiting MyD88-dependent signaling and an effect of inhibiting TLR- and IL-1R-induced cytokine activation.

Accordingly, still another aspect of the present invention relates to a MyD88 inhibitor comprising the peptide or the fusion peptide.

In the present invention, the term “inhibitor” means a molecule that partially or completely inhibits the effect of another molecule, such as a receptor or intracellular mediator, by any mechanism. Herein, “inhibitor” is used synonymously with “antagonist” or “suppressor,” and the term “inhibition” refers to a phenomenon in which biological activity or signaling activity is reduced due to deficiency, disharmony, or many other causes, and may partially or completely block, reduce, prevent, delay, inactivate, or downregulate the activity of MyD88.

In the present invention, the term “MyD88 inhibitor” refers to a substance that may directly or indirectly, or substantially, interfere with, reduce, or inhibit the biological activity of MyD88.

According to an embodiment of the present invention, the peptide inhibits the secretion of proinflammatory cytokines associated with MyD88 signaling, and thus may be efficiently utilized in a composition for preventing or treating MyD88-related diseases.

Therefore, yet another aspect of the present invention relates to a pharmaceutical composition for preventing or treating a MyD88-related disease comprising the peptide or the fusion peptide.

Still yet another aspect of the present invention relates to a method of preventing or treating a MyD88-related disease, comprising administering the peptide or the fusion peptide.

A further aspect of the present invention relates to the use of the peptide or the fusion peptide for the prevention or treatment of a MyD88-related disease.

Still a further aspect of the present invention relates to the use of the peptide or the fusion peptide for the manufacture of a medicament for the prevention or treatment of a MyD88-related inflammatory disease.

In the present invention, the MyD88-related disease is an abnormal disease caused by abnormal activation of MyD88, and is preferably an autoimmune disease, an inflammatory disease, or a neurodegenerative disease, but is not limited thereto.

In the present invention, the term “autoimmune disease” refers to a disease caused by a process in which a defect occurs in inducing or maintaining self-tolerance, thereby eliciting an immune response against self-antigens and causing the immune system to attack the body's own tissues. “Self-tolerance” refers to immunologic unresponsiveness in which the immune system does not react harmfully to antigenic substances that constitute the self. The autoimmune disease of the present invention may include, but is not limited to, psoriasis, rheumatoid arthritis, psoriatic arthritis, experimental autoimmune arthritis, asthma, Crohn's disease, multiple sclerosis, experimental autoimmune encephalomyelitis, myasthenia gravis, thyroiditis, experimental uveitis, Hashimoto's thyroiditis, primary myxedema, thyrotoxicosis, pernicious anemia, autoimmune atrophic gastritis, Addison's disease, premature menopause, male infertility, juvenile diabetes, Goodpasture's syndrome, pemphigus vulgaris, pemphigus foliaceus, sympathetic ophthalmia, phacoanaphylactic uveitis, autoimmune hemolytic anemia, idiopathic leukopenia, primary biliary cholangitis, chronic active hepatitis, cryptogenic cirrhosis, ulcerative colitis, Sjogren's syndrome, scleroderma, Wegener's granulomatosis, polymyositis, dermatomyositis, discoid lupus, or systemic lupus erythematosus.

In the present invention, the term “inflammatory disease” refers to a disease caused by an inflammatory substance (inflammatory cytokine) such as TNF-α, IL-1, IL-6, prostaglandin, leukotriene, or NO secreted from immune cells such as macrophages by excessively stimulating the immune system due to harmful stimuli such as inflammatory factors or radiation exposure. The inflammatory disease of the present invention may include, but is not limited to, insulin-dependent diabetes, eczema, allergy, atopic dermatitis, acne, atopic rhinitis, pulmonary inflammation, allergic dermatitis, chronic sinusitis, contact dermatitis, seborrheic dermatitis, gastritis, gout, gouty arthritis, ulcer, chronic bronchitis, ulcerative colitis, ankylosing spondylitis, sepsis, vasculitis, bursitis, temporal arteritis, solid tumor, arteriosclerosis, obesity, viral infection, or nonalcoholic steatohepatitis.

In the present invention, the term “neurodegenerative disease” refers to abnormalities in motor control ability, cognitive function, perception function, sensory function, and autonomic nervous system function due to a decrease or loss of nerve cell function, and is mainly classified by clinical characteristics and divided based on major symptoms and affected areas. The neurodegenerative disease of the present invention may include, but is not limited to, Alzheimer's disease, frontotemporal dementia, Lewy body dementia, corticobasal degeneration, Parkinson's disease, multiple system atrophy, Huntington's disease, progressive supranuclear palsy, Lou Gehrig's disease, primary lateral sclerosis, or spinal muscular atrophy.

As used herein, the term “prevention” means any action of inhibiting or delaying a MyD88-related disease by administering a pharmaceutical composition containing the peptide or the fusion peptide. In addition, the term “treatment” used herein means any action of alleviating or completely curing the symptoms of a MyD88-related disease by administering a pharmaceutical composition containing the peptide or the fusion peptide.

The pharmaceutical composition for preventing or treating a MyD88-related disease according to the present invention may comprise a pharmaceutically effective amount of the peptide or the fusion peptide alone, or may comprise one or more pharmaceutically acceptable carriers, excipients, or diluents. Herein, the pharmaceutically effective amount means an amount sufficient to prevent, ameliorate, and treat the symptoms of MyD88-related diseases.

The “pharmaceutically acceptable” composition refers to a composition that is physiologically acceptable and does not normally cause allergic reactions such as gastrointestinal disorders and dizziness, or similar reactions when administered to humans. Examples of such carriers, excipients, and diluents include lactose, dextrose, sucrose, sorbitol, mannitol, xylitol, erythritol, maltitol, starch, gum acacia, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil. In addition, fillers, anti-agglomeration agents, lubricants, wetting agents, fragrances, emulsifiers, and preservatives may be further included.

The term “carrier” refers to a substance that facilitates the delivery of a compound into cells or tissues.

The term “diluent” is defined as a substance that not only stabilizes the biologically active form of the compound of interest but is also diluted in water in which the compound is dissolved.

In addition, the composition of the present invention may comprise at least one known effective ingredient having a therapeutic effect on a MyD88-related disease, in addition to the peptide or the fusion peptide.

The composition of the present invention may be formulated using methods known in the art to provide rapid, sustained or delayed release of the active ingredient after administration to a mammal, including a human. The formulation may be in the form of a powder, granule, tablet, emulsion, syrup, aerosol, soft or hard gelatin capsule, sterile injectable solution, sterile powder, patch (nano patch, hydrogel patch), or microneedle.

The composition of the present invention may be administered through various routes including orally, transdermally, subcutaneously, intravenously or intramuscularly, and the dosage of the active ingredient may be appropriately selected depending on various factors such as the route of administration, the patient's age, gender, body weight, and severity of the condition, and the composition according to the present invention may be administered in combination with a known compound having an effect of preventing, ameliorating, or treating the symptoms of a MyD88-related disease.

Yet a further aspect of the present invention relates to a cosmetic composition for preventing or ameliorating a MyD88-related disease comprising the peptide or the fusion peptide.

In the cosmetic composition of the present invention, a description of the peptide or the fusion peptide may be construed in accordance with the foregoing description.

In the cosmetic composition of the present invention, the MyD88-related disease may be an autoimmune disease, an inflammatory disease, or a neurodegenerative disease, and is preferably an autoimmune disease, an inflammatory disease, or a neurodegenerative disease that may be prevented or ameliorated by the cosmetic composition, such as psoriasis, eczema, allergy, atopic dermatitis, acne, allergic dermatitis, contact dermatitis, or seborrheic dermatitis, but is not limited thereto.

The term “amelioration” of the present invention means any action that alleviates or benefits the condition of a MyD88-related disease using the peptide or the fusion peptide according to the present invention.

When the composition of the present invention is prepared into a cosmetic composition, it may comprise the peptide as an effective ingredient or components commonly used in cosmetic compositions in addition to the peptide, and examples thereof may comprise typical auxiliary agents such as antioxidants, stabilizers, solubilizers, vitamins, pigments, and fragrances, and carriers.

In the present invention, the cosmetic composition may be prepared in any formulation commonly produced in the art, and may be formulated as, for example, a solution, a suspension, an emulsion, a paste, a gel, a cream, a lotion, a powder, a soap, a surfactant-containing cleanser, an oil, a powder foundation, an emulsion foundation, a wax foundation, and a spray, but the present invention is not limited thereto. More specifically, the cosmetic composition may be prepared in the form of a nourishing cream, an astringent toner, a softening toner, a lotion, an essence, a nourishing gel, or a massage cream.

When the formulation of the cosmetic composition is a paste, a cream, or a gel, animal oil, vegetable oil, wax, paraffin, starch, gum tragacanth, cellulose derivatives, polyethylene glycol, silicone, bentonite, silica, talc, or zinc oxide may be used as a carrier component.

When the formulation of the cosmetic composition is a powder or a spray, lactose, talc, silica, aluminum hydroxide, calcium silicate, or polyamide powder may be used as a carrier component, and particularly for a spray, a propellant such as chlorofluorohydrocarbon, propane/butane or dimethyl ether may be additionally included.

When the formulation of the cosmetic composition is a solution or an emulsion, a solvent, a solubilizer, or an emulsifier is used as a carrier component, and examples thereof include water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, and fatty acid ester of sorbitan.

When the formulation of the cosmetic composition is a suspension, a liquid diluent such as water, ethanol, or propylene glycol, a suspending agent such as ethoxylated isostearyl alcohol, polyoxyethylene sorbitol ester, or polyoxyethylene sorbitan ester, microcrystalline cellulose, aluminum metahydroxide, bentonite, agar, or tragacanth may be used as a carrier component.

When the formulation of the cosmetic composition is a surfactant-containing cleanser, aliphatic alcohol sulfate, aliphatic alcohol ether sulfate, sulfosuccinic acid monoester, isethionate, imidazolinium derivative, methyl taurate, sarcosinate, fatty acid amide ether sulfate, alkylamidobetaine, fatty alcohol, fatty acid glyceride, fatty acid diethanolamide, vegetable oil, lanolin derivative, or ethoxylated glycerol fatty acid ester may be used as a carrier component. In the present invention, the cosmetic composition may be formulated by stabilizing the peptide or the fusion peptide by incorporating the same into a nanoliposome. By incorporating the peptide or the fusion peptide into the nanoliposome, the components of the peptide or the fusion peptide are stabilized, thereby resolving problems such as precipitation and deformation during formulation, and increasing the solubility and percutaneous absorption rate of the components, ultimately maximizing the efficacy expected from the peptide or the fusion peptide.

The cosmetic composition of the present invention may also be in the form of a polymer gel or a microneedle made of a polymer, in which the polymer is starch, cellulose ether, carboxymethyl cellulose, hydroxypropyl methylcellulose, hydroxyethyl cellulose, ethylhydroxyethyl cellulose, alginate, carrageenan, hyaluronic acid and derivatives thereof, polyacrylic acid, polysulfonate, or the like.

Hereinafter, the present invention will be described in more detail with reference to examples. However, it will be obvious to those skilled in the art that these examples are provided only for illustration of the present invention and should not be construed as limiting the scope of the present invention.

### Example 1: Materials and Methods

### Example 1-1: Peptide Design, Optimization, and Synthesis

To design peptides that may bind to MyD88, the Protein Data Bank (PDB) files for MyD88 (PDB ID: 7BEQ) and MAL (PDB ID: 5UZB) were downloaded and prepared for analysis by removing all missing residues, gaps, or structural issues. PyRosetta (Sidhartha Chaudhury, et al., PyRosetta: Bioinformatics. 2010 Mar. 1; 26(5):689-9), a Python-based protein design software package, was used for peptide design and optimization. The interface region of MyD88 and interacting proteins were analyzed using PyRosetta's protein-protein coupling capabilities, which predict potential binding orientations and interactions between proteins. Suitable peptide templates were selected using the PyRosetta fragment insertion method (Jianyi Yang, et al., Proc Natl Acad Sci USA. 2020 Jan. 21; 117(3):1496-1503) from existing databases or through de novo structure generation. A set of peptide conformations was generated from a selected template structure that allowed mutations, insertions, or deletions. Undesirable conformations were filtered out through energy minimization and a scoring function. Optimal peptides were selected and improved through repacking, minimization, and side-chain optimization, and the energy and stability thereof were evaluated using scoring functions (Rebecca F Alford, et al., J Chem Theory Comput. 2017 Jun. 13; 13(6):3031-3048). The most promising peptide structures were selected for experimental validation based on the structural energy, stability, and compatibility with the MyD88 protein structure.

An alanine scanning method was used to design potent derivatives of experimentally effective peptides. Each residue in the initial peptide was mutated to alanine one at a time, leaving the remaining residues unchanged. To identify essential residue(s) for peptide stability and function, the energies of mutant peptides were evaluated using PyRosetta's scoring function. These key residues were mutated and replaced with other amino acids, and new peptides with altered properties were designed. To identify the most promising candidates, the designed peptides were scored for energy and stability using PyRosetta's scoring function. The selected peptides were further refined through repacking, minimization, and side-chain optimization, and then finally evaluated for energy and stability. To facilitate intracellular delivery, penetratin (RQIKIWFQNRRMKWKK (SEQ ID NO: 12)) as a CPP was fused to the N-terminus of the peptide (D Derossi, et al., Journal of Biological Chemistry, 1994 Apr. 8; 269(14):10444-50). The peptide was synthesized by Peptron (Daejeon, Korea) with a purity of >90%, and the molecular weight thereof was determined using liquid chromatography-mass spectrometry.

### Example 1-2: Chemicals and Reagents

IL-1R protein was purchased from Bio-Techne (Lille, France). Lipopolysaccharide (LPS; Escherichia coli 0111: B4) was purchased from Sigma-Aldrich (St. Louis, MO, USA), and Pam₃CSK₄, poly(I:C), imiquimod (IMQ), resiquimod (R848), and CpG oligodeoxynucleotide (CpG-ODN) were purchased from Thermo Fisher Scientific, Inc. (Waltham, MA, USA). FSL-1 was purchased from InvivoGen (San Diego, CA, USA).

Antibodies against phospho-(p-)p65, p-JNK, JNK, ERK, p-p38, p38, and Iκ-Bα were purchased from Cell Signaling Technology Inc. (Danvers, MA, USA), and antibodies against p-ERK and β-actin were purchased from Santa Cruz Biotechnology Inc. (Dallas, TX, USA).

### Example 1-3: Peptide Dissolution

A 10 mM stock solution was prepared by dissolving the peptide in sterile water and frozen at −20 °C until use. MyDIP2 and MyDIP2-4 have purity of 98% and 99%, respectively. For in vivo analysis of psoriasis and EAE, MyDIP2-4 was dissolved in phosphate-buffered saline (PBS). The same solvent was added to the control and disease groups.

### Example 1-4: Cell Culture

THP-1 and RAW 264.7 cells (ATCC, Manassas, VA, USA) were cultured at 37 °C and 5% CO₂ in RPMI (Roswell Park Memorial Institute) medium and Dulbecco's modified Eagle medium supplemented with fetal bovine serum (FBS, 10%), 4 mM L-glutamine, 1 mM sodium pyruvate, penicillin (100 units/ml), and streptomycin (0.1 mg/ml). THP-1 cells, which are monocytes, were differentiated into macrophages by treatment with 80 nM phorbol 12-myristate 13-acetate (PMA, Sigma-Aldrich) for 24 hours. The cells were cultured at 37 °C and 5% CO₂ in a humidified incubator (Thermo Fisher Scientific, Inc.), and the medium was replaced every 18 hours.

### Example 1-5: Measurement of Macrophage Viability

To evaluate the cytotoxic effect of the peptide on mouse and human macrophages, RAW 264.7 cells and THP-1-derived macrophages were seeded at respective densities of 5×10⁴/well and 2×10⁵/well in 96-well plates (BD Biosciences, San Jose, CA, USA) followed by culture overnight at 37 °C in a humidified incubator. After treating the cells with the peptide at the corresponding concentration for 24 hours, viability was measured using 3-(4,5-dimethylthiazol-2-yl)-2,5-diphenyltetrazolium bromide (MTT) assay (Sigma-Aldrich). After 24 hours of treatment, the medium was removed, 100 μl of MTT solution was added to each well, and the plate was incubated at 37 °C for 3 hours. Next, the MTT solution was gently removed by suction, 100 μl of dimethyl sulfoxide was added to each well, and the plate was slowly shaken to dissolve the tetrazolium dye and then incubated at 37 °C for 30 minutes. Relative cell viability was calculated by measuring absorbance at 570 nm, and the untreated control group was designated as having a relative viability of 100%. Cell viability in different groups was calculated by comparing the same with the corresponding value in the control group.

### Example 1-6: Enzyme-Linked Immunosorbent Assay (ELISA)

To evaluate the inhibitory effect of the peptide on cytokine secretion in response to various stimuli, RAW 264.7 cells and THP-1-derived macrophages were seeded at respective densities of 5×10⁴/well and 2×10⁵/well in 96-well plates (BD Biosciences) followed by culture overnight. After 24 hours of treatment, IL-6 secretion was assessed using a mouse IL-6 ELISA kit (Invitrogen). Tumor necrosis factor (TNF)-α production was assessed after 4 hours of treatment using a mouse TNF alpha ELISA kit (Invitrogen) according to the manufacturer's protocol. For THP-1-derived macrophages, the production of TNF-α and IL-6 was measured using human TNF-α and IL-6 ELISA kits (Invitrogen), respectively. Absorbance was measured at the appropriate wavelength using a microplate spectrophotometer system (Synergy HTX Multimode Reader, BioTek Instruments; Winooski, VT, USA).

### Example 1-7: Protein Quantification and Western Blot Analysis

For total protein extraction, Mammalian Protein Extraction Reagent (M-PER, Thermo Fisher Scientific, Inc.) was used, and a bicinchoninic acid (BCA) assay kit (Sigma-Aldrich) was used to evaluate protein concentration. Western blot analysis, including gel electrophoresis and transfer, was performed using the Mini-PROTEAN Tetra Cell and Mini Trans-Blot Electrophoretic Transfer Cell System (Bio-Rad Laboratories, Hercules, CA, USA). The membranes were diluted 1:1000 with antibodies against p-p65, p-JNK, JNK, p-ERK, ERK, p-p38, p38, IκBα, and β-actin and incubated overnight at 4 °C. The membranes were washed vigorously with PBS containing 0.1% Tween 20 (PBST, PBS+0.1% Tween 20) and incubated with peroxidase-conjugated anti-mouse or anti-rabbit IgG antibody (1:1000) for 2 hours. Proteins were detected with SuperSignal West Pico ECL solution (Thermo Fisher Scientific, Inc.) and visualized with a ChemiDoc Touch Imaging System (Bio-Rad Laboratories).

### Example 1-8: Animal Testing

### Example 1-8-1: Evaluation of Efficacy of MyDIP2-4 in Experimental Autoimmune Encephalomyelitis (EAE) Model

### (1) Mouse

Eight-week-old female C57BL/6 (B6) mice (Orient Bio, Korea) were maintained in a specific pathogen-free environment on a standard laboratory ad libitum diet. All experiments were approved by the Animal Care and Use Committee [NDIC Approval Nos. 12022111 (EAE) and I2022053-3 (psoriasis), Hwaseong, Korea] and were performed in accordance with relevant guidelines and regulations.

### (2) EAE Induction and Drug Administration

EAE was induced in mice using the Hooke Kit (MOG₃₅₋₅₅/complete Freund's adjuvant (CFA) Emulsion PTX (Hooke Laboratories, LLC, Lawrence, MA, USA)) according to the manufacturer's protocol. Mice were immunized subcutaneously with myelin oligodendrocyte glycoprotein emulsified in complete Freund's adjuvant. Pertussis toxin (110 ng) was injected intraperitoneally at the time of immunization and 24 hours later. Dimethyl fumarate (100 mg/kg), a positive control group, was administered orally, and MyDIP2-4 peptide was administered intraperitoneally at a dose of 40 mg/kg once daily from the second day until the day of sacrifice.

### (3) Behavioral Assessment

Mice were weighed and examined daily, and EAE symptoms were assessed using the following standard scoring system: 0-6: 0, no symptoms; 1, limp tail; 2, limp tail and hind limb paralysis; 3, paralysis of one or more hind limbs; 4, complete hind limb and hindquarter paralysis; 5, hindquarter paralysis with forelimb paralysis; and 6, death. The blood was collected from animals for cytokine analysis after euthanasia.

### Example 1-8-2: Evaluation of Efficacy of MyDIP2-4 in Imiquimod-Induced Psoriasis Model

### (1) Mouse

Six-week-old female C57BL/6 mice (Orient Bio, Seongnam, Korea) were housed under specific pathogen-free conditions on a standard laboratory ad libitum diet. Animal experiments related to psoriasis were approved by the Animal Care and Use Committee [NDIC Approval Nos. 12022111 (EAE) and I2022053-3 (psoriasis), Hwaseong, Korea] and were performed in accordance with relevant guidelines and regulations.

### (2) IMQ-Induced Psoriasis Induction and Drug Treatment

Disease was induced by topical application of Aldara cream (Donga ST; Seoul, Korea) containing 5% imiquimod onto the back of six-week-old female C57BL/6 mice for 5 consecutive days. MyDIP2-4 was administered intraperitoneally at doses of 5 and 20 mg/kg. As a positive control group, dexamethasone (Sigma-Aldrich; cat. No. D1756) was administered orally at a dose of 10 mg/kg. Five days later, mice were sacrificed under respiratory anesthesia, and skin lesions were collected for histological examination.

### (3) Evaluation of Psoriasis Area and Severity Index (PASI)

An objective scoring system to quantify the severity of back skin inflammation was established based on the PASI scoring system, a widely used clinical tool to assess psoriasis severity. On a scale from 0 to 4, factors such as erythema, scaling, wrinkles, and thickness were individually assessed as follows: 0, none; 1, weak; 2, moderate; 3, significant; and 4, very evident. Under the careful supervision of experienced investigators, erythema, scaling, and thickness were graded. The cumulative score (erythema+scaling+thickness score, scale 0-12) indicates the severity of psoriasis symptoms.

### (4) H&E (Hematoxylin and Eosin) Staining and Immunohistochemistry (IHC)

Skin tissue was removed, the upper layer was fixed in neutral formalin, and H&E staining and IHC were performed. Epidermal thickness of skin tissue was measured using H&E staining. IL-17, KI-67, and IL-23 levels were measured using IHC with the aid of antibodies against IL-17A (Thermo Fisher Scientific; cat. No. PA5-79470), KI-67 (Cell Signaling; cat. No. 12202), and IL-23 (Novus Biologicals; cat. No. NBP1-76697).

### Example 1-9: Surface Plasmon Resonance (SPR) Analysis

The biophysical interactions between MyDIP2-4 and MyD88 were evaluated using SPR analysis in an iMSPR-mini system (icluebio, Seongnam, Korea). MyD88 protein was immobilized on the surface of a nitrilotriacetic acid (NTA) sensor chip at a concentration of 50 μg/ml. MyDIP2-4 was injected into the MyD88-binding chip at various concentrations ranging from 0.8 to 12.8 μM (dissolved in PBS) at a flow rate of 50 μl/min. PBS was used as a running buffer and injected into the empty channel as a reference. Experiments were performed twice using freshly prepared reagents, and data were analyzed using TraceDrawer software (Ridgeview Instruments; Uppsala, Sweden).

### Example 1-10: Statistical Analysis

All experiments were randomized and blinded. All experiments were performed independently at least three times, and the data obtained were analyzed using two-tailed t-test or one-way analysis of variance (ANOVA) with GraphPad Prism 7 (GraphPad Software, Inc., San Diego, CA, USA).

### Example 2: Identification of MyDIP2 as MyD88-Mediated Signaling Inhibitor

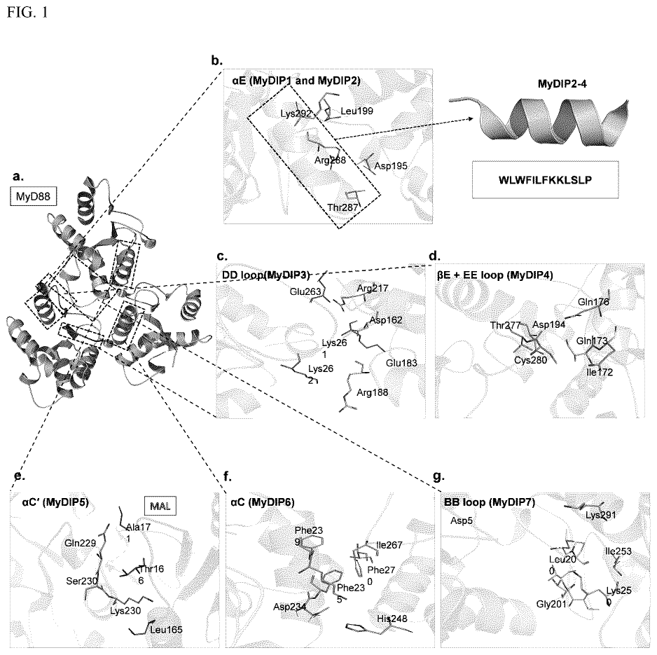

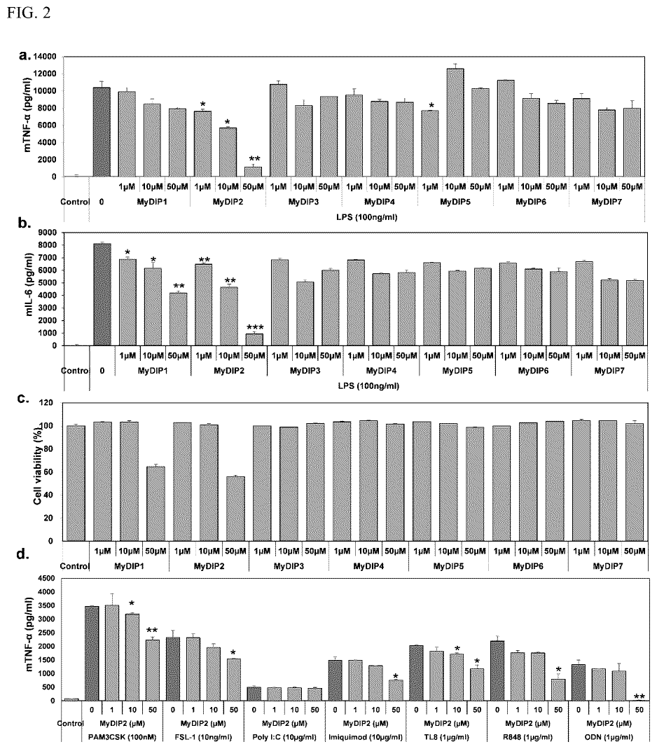

MyD88 plays a crucial role in activating the immune response and recognizes pathogenic microorganisms and danger signals through interactions with proteins such as TIRAP, TLR4, and IRAK4. As demonstrated in previous studies, disrupting the TIR-TIR domain interaction of MyD88 through targeted substitution of specific residues may interfere with downstream signaling and potentially treat autoimmune diseases (Thomas Ve, et al., Nat Struct Mol Biol. 2017 September; 24(9):743-751). Using the PyRosetta program, peptides were designed (FIG. 1) to target this domain and disrupt the interaction, and the activity thereof toward MyD88 was detected by LPS-induced activation of TLR4. The corresponding peptides and interaction interfaces are shown in Table 2. LPS activates NF-κB through MyD88-dependent signaling, leading to the secretion of proinflammatory cytokines including TNF and IL-6 (Hongli Zhang, et al., Food and Chemical Toxicology, 2021 January; 147:111915). When tested for LPS stimulation, MyDIP1 and MyDIP2 significantly inhibited IL-6 secretion, but only MyDIP2 significantly downregulated TNF-α secretion in response to LPS stimulation. Thus, of all the peptides tested for LPS stimulation, MyDIP2 was shown to maximally inhibit LPS-mediated TNF-α and IL-6 activation in mouse macrophages (RAW 264.7 cells) (FIGS. 2a and 2b). A CPP had no inhibitory effect on LPS-induced TNF-α secretion (Masaud Shah, et al., Biomaterials. 2020 July; 245:119974). After 24 hours of each peptide treatment, the toxicity of the peptides was evaluated at concentrations ranging from 1 to 50 μM using colorimetric 3-(4,5-dimethylthiazol-2-yl)-2,5-diphenyltetrazolium bromide (MTT) assay in RAW 264.7 cells (FIG. 2c).

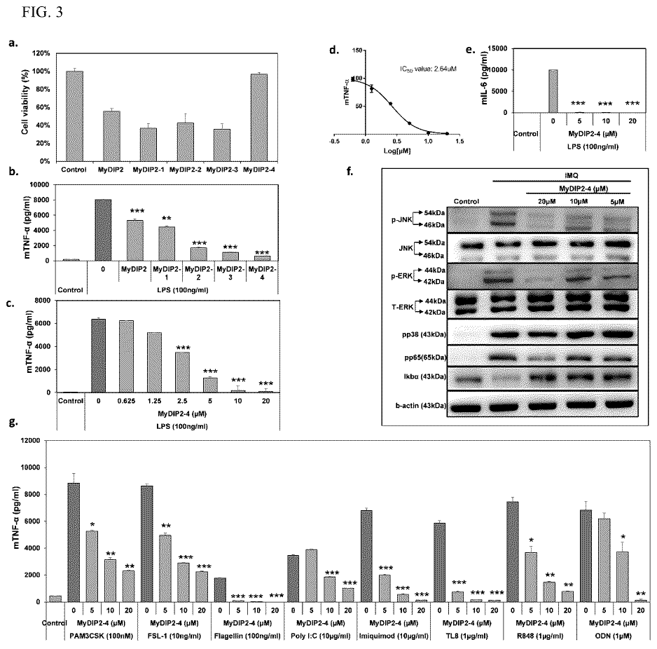

The inhibitory effect of MyDIP2 on all TLR ligands except TLR3 was evaluated based on the fact that MyD88 functions as an adaptor protein in all TLR-mediated signaling pathways. MyDIP2 inhibited TNF-α secretion in response to all tested TLRs, including TLR1/2, TLR2/6, TLR5, TLR7, TLR7/8, and TLR9, but not TLR3. This suggests that MyDIP2 functions as an inhibitor of MyD88, which is involved in all TLRs except TLR3 (FIG. 2d). To enhance the inhibitory potency of MyDIP2, the peptide was further modified to design four additional peptides. The cytotoxicity of the four peptides was tested using MTT assay at a concentration of 50 μM, and the efficacy thereof against LPS (100 ng/ml) was tested using ELISA at 20 μM. The data revealed that all four peptides exhibited superior TNF-α inhibitory activity compared to MyDIP2. Among the tested peptides, MyDIP2-4 exhibited the highest efficacy. MyDIP2-4 was not cytotoxic to mouse macrophages (FIGS. 3a and 3b).

TABLE 2
Identification and characterization of
peptide sequences and target domains

| | | SEQ | | |
|---|---|---|---|---|
| | | ID | Sub- | Target |
| Name | Sequence | NO: | domain | domain |
| MyDIP1 | WFWTRLAKA | 1 | Helix αE | MAL/TIR |
| MyDIP2 | WFWTRLAKALSLP | 2 | Helix αE | MyD88 |
| MyDIP3 | KYKAMKKE | 3 | DD loop | MyD88 |
| MyDIP4 | DYTNPCTKS | 4 | βE + EE | MyD88 |
| | | | loop | |
| MyDIP5 | SDDYLQSK | 5 | Helix αC′ | MyD88 |
| MyDIP6 | ECDFQTMFALSL | 6 | Helix αC | MyD88 |
| MyDIP7 | SDRDVLPGTCVWS | 7 | BB loop | DD/EE |
| | IA | | | loop |
| | | | | MyD88 |

MyDIP2 derivatives

| | | | | |
|---|---|---|---|---|
| MyDIP2-1 | WFWYRLAKKLSLP | 8 | Helix αE | MyD88 |
| MyDIP2-2 | WFWYRLIKKLSLP | 9 | Helix αE | MyD88 |
| MyDIP2-3 | WFWYILLKKLSLP | 10 | Helix αE | MyD88 |
| MyDIP2-4 | WLWFILFKKLSLP | 11 | Helix αE | MyD88 |

### Example 3: Inhibitory Effect of MyDIP2-4 on MyD88-Mediated TLR Activation and Cytokine Secretion in Mouse Macrophages

To determine the effect of MyDIP2-4 on RAW 264.7 cells, the effect of MyDIP2-4 on IL-6 secretion was investigated. To this end, cells were pretreated with various doses of MyDIP2-4 and then treated with LPS. IL-6 levels were quantified by ELISA. MyDIP2-4 significantly inhibited LPS-mediated IL-6 secretion (FIG. 3e). IC₅₀ values were calculated by measuring the levels of TNF-α secreted from RAW 264.7 cells in response to LPS stimulation. The IC₅₀ value of MyDIP2-4 for LPS stimulation was 2.6 μM (FIGS. 3c and 3d).

Since MyD88 is involved in signaling of various TLRs, the effect of MyDIP2-4 on TNF-α secretion levels in RAW 264.7 cells in response to different ligands, including Pam₃CSK₄ (TLR1/2), FSL-1 (TLR2/6), poly(I:C) (TLR3), flagellin (TLR5), resiquimod (R848, TLR7/8), TL8 (TLR8), or CpG-ODN (TLR9), was measured by ELISA. Significant inhibition of TLR signaling was observed for all ligands (FIG. 3g).

### Example 4: Inhibitory Effect of MyDIP2-4 on TLR7-Dependent NF-κB Signaling and MAPK Expression

To determine the effect of MyDIP2-4 on the protein expression of NF-κB and MAPKs, Western blot analysis was performed. MyDIP2-4 was found to significantly downregulate phospho p-65 expression and block the degradation of Iκ-Bα. Additionally, MyDIP2-4 inhibited MAPK phosphorylation, including ERK, JNK, and p-p38 (FIG. 3f).

### Example 5: Downregulation of TLR/IL-1R-Mediated Signaling by MyDIP2-4 in Human Macrophages

The effects of MyDIP2 and MyDIP2-4 on THP-1-derived macrophages were evaluated. The inhibitory effect of different concentrations of MyDIP2-4 on THP-1-derived macrophages was evaluated upon LPS stimulation. ELISA results showed that MyDIP2-4 inhibited TNF-α secretion in THP-1-derived macrophages more efficiently than MyDIP2 (FIGS. 4b and 4c). To investigate the effect of MyDIP2-4 on IL-1R signaling, TNF-α secretion was assessed in THP-1-derived macrophages pretreated with MyDIP2-4 peptide and then treated with IL-1β protein. MyDIP2-4 efficiently inhibited IL-1R signaling in THP-1-derived macrophages (FIG. 4d).

To confirm the specificity of MyDIP2-4, IL-1β levels were measured as a result of NLRP3 activation following nigericin treatment. To this end, PMA-differentiated THP-1 cells were primed with LPS (TLR4 ligand) and then treated with MyDIP2-4 peptide, after which NLRP3 was activated with nigericin. Although MyDIP2-4 was found to downregulate TNF-α in response to LPS, no effect was observed on nigericin-activated IL-1β levels (FIG. 4e).

### Example 6: Effect of MyDIP2-4 on Myelin Oligodendrocyte Glycoprotein (MOG₃₅₋₅₅)-Induced EAE

To investigate the therapeutic potential of MyDIP2-4 on EAE progression, mice were immunized with MOG₃₅₋₅₅ peptide in CFA and pertussis toxin was administered once on day 0 and again 24 hours later. Mice were administered MyDIP2-4 (40 mg/kg) or dimethyl fumarate (DMF, 100 mg/kg) intraperitoneally once daily. Onset was observed on day 6. Mouse body weight and EAE clinical scores were recorded three times a week: days 0, 2, 5, 7, 9, 12, 14, 16, 19, and 21. As a result, MyDIP2-4-treated mice showed no difference in body weight compared to DMF-treated mice (FIG. 4i). At day 21, DMF reduced clinical scores compared to MyDIP2-4 (FIG. 4h). In contrast, MyDIP2-4 was confirmed to significantly inhibit IL-6 and IFN-γ cytokine secretion (FIGS. 4l to 4m).

### Example 7: Evaluation of Therapeutic Potential of MyDIP2-4 in IMQ-Induced Psoriasis

To establish an IMQ-induced psoriasis model, Aldara cream containing 5% imiquimod was topically applied onto the back of six-week-old female C57BL/6 mice for 5 consecutive days. Dexamethasone as a positive control group was administered orally, and MyDIP2-4 was administered intraperitoneally. The doses of MyDIP2-4 were 5 mg/kg and 20 mg/kg, and the dose of dexamethasone was 10 mg/kg. PBS was used as a vehicle control group. The data suggested that progressive symptoms may be observed in the IMQ administration group (FIG. 5c). The epidermal thickness was significantly reduced in the dexamethasone-treated group, but not in the MyDIP2-4-treated group (FIG. 5e). Immunohistochemical data suggested a significant decrease in the proportion of KI-67-positive cells in both MyD88- and dexamethasone-treated groups. Additionally, MyDIP2-4 significantly reduced IL-23 levels (FIG. 5f). Dexamethasone and MyDIP2-4 did not inhibit the expression of IL-17 (FIG. 5f).

### Example 8: Analysis of Biophysical Interaction Between MyDIP2-4 and MyD88 by SPR

SPR technology was used to evaluate the biophysical interaction between the MyDIP2-4 peptide and the MyD88 protein. SPR analysis involves immobilizing the MyD88 protein on the surface of a sensor chip and then flowing the MyDIP2-4 peptide at various concentrations. SPR sensorgrams showed that the MyDIP2-4 peptide bound to the immobilized MyD88 protein in a concentration-dependent manner (FIG. 4f). Additionally, the rate constants for association (Ka) and dissociation (Kd) for the MyDIP2-4-MyD88 interaction were measured and found to be 3.70 M⁻¹s⁻¹ and 1.37×10⁻⁷ s⁻¹, respectively. Overall, the data support the notion that the MyDIP2-4 peptide exhibits a strong and dose-dependent interaction with human MyD88 protein.

### INDUSTRIAL APPLICABILITY

According to the present invention, it was demonstrated through in vitro and in vivo experiments that candidate peptides targeting MyD88 are potent inhibitors capable of effectively suppressing TLR- and IL-1R-mediated signaling pathways. Peptides can be a promising option for therapeutic development because they offer better efficacy, safety, selectivity, and potential compared to small molecules and chemicals. Among the candidates targeting MyD88, MyDIP2-4 (MyD88 inhibitory peptide 2-4) appears to be the most effective and potent peptide, and thus MyDIP2-4 can be an effective therapeutic agent for treating autoimmune complications caused by pathogens or environmental factors.

Having described certain parts of the present invention in detail above, it will be obvious to those skilled in the art that these specific descriptions are only preferred embodiments, and the scope of the present invention is not limited thereby. Accordingly, the substantial scope of the present invention will be defined by the appended claims and equivalents thereto.

### SEQUENCE LISTING FREE TEXT

An electronic file is attached.

1. A peptide represented by any one amino acid sequence selected from the group consisting of SEQ ID NO: 1 to SEQ ID NO: 11.

2. The peptide according to claim 1, wherein the peptide inhibits MyD88-mediated signaling and thereby suppresses TLR- and IL-1R-mediated signaling pathways.

3. A fusion peptide in which the peptide according to claim 1 is bound to a cell-penetrating peptide (CPP).

4. A MyD88 inhibitor comprising the peptide according to claim 1 or a fusion peptide in which the peptide according to claim 1 is bound to a cell-penetrating peptide (CPP).

5. A pharmaceutical composition for preventing or treating a MyD88-related disease, comprising the peptide according to claim 1 or a fusion peptide in which the peptide according to claim 1 is bound to a cell-penetrating peptide (CPP).

6. The pharmaceutical composition according to claim 5, wherein the MyD88-related disease is an autoimmune disease, an inflammatory disease, or a neurodegenerative disease.

7. The pharmaceutical composition according to claim 6, wherein the autoimmune disease is psoriasis, rheumatoid arthritis, psoriatic arthritis, experimental autoimmune arthritis, asthma, Crohn's disease, multiple sclerosis, experimental autoimmune encephalomyelitis, myasthenia gravis, thyroiditis, experimental uveitis, Hashimoto's thyroiditis, primary myxedema, thyrotoxicosis, pernicious anemia, autoimmune atrophic gastritis, Addison's disease, premature menopause, male infertility, juvenile diabetes, Goodpasture's syndrome, pemphigus vulgaris, Pemphigus foliaceus, sympathetic ophthalmia, phacoanaphylactic uveitis, autoimmune hemolytic anemia, idiopathic leukopenia, primary biliary cholangitis, chronic active hepatitis, cryptogenic cirrhosis, ulcerative colitis, Sjogren's syndrome, scleroderma, Wegener's granulomatosis, polymyositis, dermatomyositis, discoid lupus, or systemic lupus erythematosus.

8. The pharmaceutical composition according to claim 6, wherein the inflammatory disease is insulin-dependent diabetes, eczema, allergy, atopic dermatitis, acne, atopic rhinitis, pulmonary inflammation, allergic dermatitis, chronic sinusitis, contact dermatitis, seborrheic dermatitis, gastritis, gout, gouty arthritis, ulcer, chronic bronchitis, ulcerative colitis, ankylosing spondylitis, sepsis, vasculitis, bursitis, temporal arteritis, solid tumor, arteriosclerosis, obesity, viral infection, or nonalcoholic steatohepatitis.

9. The pharmaceutical composition according to claim 6, wherein the neurodegenerative disease is Alzheimer's disease, frontotemporal dementia, Lewy body dementia, corticobasal degeneration, Parkinson's disease, multiple system atrophy, Huntington's disease, progressive supranuclear palsy, Lou Gehrig's disease, primary lateral sclerosis, or spinal muscular atrophy.

10. A cosmetic composition for preventing or ameliorating a MyD88-related disease, comprising the peptide according to claim 1 or a fusion peptide in which the peptide according to claim 1 is bound to a cell-penetrating peptide (CPP).

11. The cosmetic composition according to claim 10, wherein the MyD88-related disease is an autoimmune disease, an inflammatory disease, or a neurodegenerative disease.


SEQUENCE LISTING (25 sequences)

SEQ ID NO: 1 - protein, 9 aa, synthetic construct
WFWTRLAKA

SEQ ID NO: 2 - protein, 13 aa, synthetic construct
WFWTRLAKALSLP

SEQ ID NO: 3 - protein, 8 aa, synthetic construct
KYKAMKKE

SEQ ID NO: 4 - protein, 9 aa, synthetic construct
DYTNPCTKS

SEQ ID NO: 5 - protein, 8 aa, synthetic construct
SDDYLQSK

SEQ ID NO: 6 - protein, 12 aa, synthetic construct
ECDFQTMFALSL

SEQ ID NO: 7 - protein, 15 aa, synthetic construct
SDRDVLPGTCVWSIA

SEQ ID NO: 8 - protein, 13 aa, synthetic construct
WFWYRLAKKLSLP

SEQ ID NO: 9 - protein, 13 aa, synthetic construct
WFWYRLIKKLSLP

SEQ ID NO: 10 - protein, 13 aa, synthetic construct
WFWYILLKKLSLP

SEQ ID NO: 11 - protein, 13 aa, synthetic construct
WLWFILFKKLSLP

SEQ ID NO: 12 - protein, 16 aa, synthetic construct
RQIKIWFQNRRMKWKK

SEQ ID NO: 13 - protein, 11 aa, synthetic construct
YGRKKRRQRRR

SEQ ID NO: 14 - protein, 13 aa, synthetic construct
GRKKRRQRRRPPQ

SEQ ID NO: 15 - protein, 9 aa, synthetic construct
RKKRRQRRR

SEQ ID NO: 16 - protein, 8 aa, synthetic construct
RRRRRRRR

SEQ ID NO: 17 - protein, 13 aa, synthetic construct
GRRRRRRRRRPPQ

SEQ ID NO: 18 - protein, 21 aa, synthetic construct
KETWWETWWTEWSQPKKKRKV

SEQ ID NO: 19 - protein, 11 aa, synthetic construct
YARVRRRGPRR

SEQ ID NO: 20 - protein, 18 aa, synthetic construct
KLALKLALKALKAALKLA

SEQ ID NO: 21 - protein, 18 aa, synthetic construct
LLIILRRRIRKQAHAHSK

SEQ ID NO: 22 - protein, 27 aa, synthetic construct
GALFLGFLGAAGSTMGAWSQPKKKRKV

SEQ ID NO: 23 - protein, 27 aa, synthetic construct
GWTLNSAGYLLGKINLKALAALAKKIL

SEQ ID NO: 24 - protein, 20 aa, synthetic construct
HGLASTLTRWAHYNALIRAF

SEQ ID NO: 25 - protein, 17 aa, synthetic construct
NAKTRRHERRRKLAIER